SEQUENCE LISTING (10 sequences)

SEQ ID NO: 1 - protein, 40 aa, Pseudoplectania nigrella
GFGCNGPWDEDDMQCHNHCKSIKGYKGGYCAKGGFVCKCY

SEQ ID NO: 2 - protein, 44 aa, Heliothis virescens
  MISC_FEATURE (1)..(44): SEQ ID NO2 of WO 99/53053
DKLIGSCVWGAVNYTSDCNGECKRRGYKGGHCGSFANVNCWCET

SEQ ID NO: 3 - protein, 41 aa, Aspergillus nidulans
  MISC_FEATURE (1)..(41): SEQ ID NO1 of WO 02/085934
SSCQLGGIFGAGDAACSASCIRAGTYHGGYCNDKQVCICTH

SEQ ID NO: 4 - protein, 90 aa, Eurotium amstelodami
  MISC_FEATURE (1)..(90): SEQ ID NO2 of US 60/629,442
MHFTKVSTILFTIFAAGIMAAPTEGVREEAAPGQEVYPDEPPASLTKRGFGCPGDAYQCSEHCRALGGGRTGGYCAGPWYLGHPTCTCSF

SEQ ID NO: 5 - protein, 90 aa, Picea glauca
  MISC_FEATURE (1)..(90): SEQ ID NO2 of US 60/632,672
MKFTISIIAALAFFAQGIVAAPAPIPEAAAVAAPEAEPKALDELPELQKRGFGCNGWPFEDDEQCHNHCKTIPGYKGGYCANVGTTCKCY

SEQ ID NO: 6 - protein, 62 aa, Crassostrea virginica
  MISC_FEATURE (1)..(62): SEQ ID NO2 of US 60/632,673
MKVFVLLTIAVMLMVSADVALAGFGCPLNRYQCHSHCQSIGRKGGYCGGWWSFTCTCYRTKK

SEQ ID NO: 7 - protein, 62 aa, Mesobuthus gibbosus
  MISC_FEATURE (1)..(62): SEQ ID NO2 of US 60/632,670
MKTIVLLFVLALVFCTLEMGMVEAGFGCPFNQGRCHRHCRSIRRRGGYCDGFLKQRCVCYRR

SEQ ID NO: 8 - protein, 64 aa, Crassostrea gigas
  MISC_FEATURE (1)..(64): SEQ ID NO2 of US 60/632,669
MKVFVLLTLAVLLMVSADMAFAGFGCPGNQLKCNNHCKSISCRAGYCDAATLWLRCTCTDCNGK

SEQ ID NO: 9 - protein, 64 aa, Crassostrea virginica
  MISC_FEATURE (1)..(64): SEQ ID NO2 of US 60/632,486
MKVFVLLTIAVMLLVSADVATADNGCPRRPRICHNRCIYKGRRGGKCVGKWRSLCECIYPSKAG

SEQ ID NO: 10 - protein, 69 aa, Aspergillus oryzae
  MISC_FEATURE (1)..(69): SEQ ID NO2 of US 60/642,076
MKLLTVAFSLLLLGQVHASPLVLDKRSSCQLGDVWDLNAADAACSASCAIQHGDKHGGHCDKNKVCVCN

(19) United States
(12) Patent Application Publication
(10) Pub. No.: US 2006/0217306 A1
(43) Pub. Date: Sep. 28, 2006

(54) Systemic treatment of infections with defensins

(75) Inventors: Hans-Henrik Kristensen, Holte (DK); Per Holse Mygind, Soeborg (DK); Dorotea Raventos Segura, Humlebaek (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 11/350,321

(22) Filed: Feb. 8, 2006

Related U.S. Application Data
(60) Provisional application No. 60/650,878, filed on Feb. 8, 2005.

(51) Int. Cl. A61K 38/17

(52) U.S. Cl. 514/12

(57) ABSTRACT

The present invention relates to methods for treating systemic microbial infections with defensin antimicrobial peptides. The invention also relates to a medicament and a method for preparing a medicament.

### CROSS-REFERENCE TO RELATED APPLICATIONS

This application claims priority or the benefit under 35 U.S.C. 119 of U.S. provisional application No. 60/650,878 filed Feb. 8, 2005, the contents of which are fully incorporated herein by reference.

### FIELD OF THE INVENTION

The present invention relates to systemic treatment of microbial infections with defensin antimicrobial peptides.

### BACKGROUND OF THE INVENTION

It is an object of the present invention to provide methods for systemic treatment of microbial infections, such as bacterial, viral and fungal infections, with defensin antimicrobial peptides, which are non-toxic and which are degraded very slowly in human blood serum.

### SUMMARY OF THE INVENTION

The present invention relates to a method for treating a microbial infection, comprising contacting a microbial population systemically with a defensin having antimicrobial activity.

In a second aspect, the invention relates to a pharmaceutical formulation for systemic treatment of a microbial infection.

In a third aspect the invention relates to a method for preparing the pharmaceutical formulation.

Definitions

Antimicrobial activity: The term “antimicrobial activity” is defined herein as an activity which is capable of killing or inhibiting growth of microbial cells. In the context of the present invention the term “antimicrobial” is intended to mean that there is a bactericidal and/or a bacteriostatic and/or fungicidal and/or fungistatic effect and/or a virucidal effect, wherein the term “bactericidal” is to be understood as capable of killing bacterial cells. The term “bacteriostatic” is to be understood as capable of inhibiting bacterial growth, i.e. inhibiting growing bacterial cells. The term “fungicidal” is to be understood as capable of killing fungal cells. The term “fungistatic” is to be understood as capable of inhibiting fungal growth, i.e. inhibiting growing fungal cells. The term “virucidal” is to be understood as capable of inactivating virus. The term “microbial cells” denotes bacterial or fungal cells (including yeasts).

In the context of the present invention the term “inhibiting growth of microbial cells” is intended to mean that the cells are in the non-growing state, i.e., that they are not able to propagate.

For purposes of the present invention, antimicrobial activity may be determined according to the procedure described by Lehrer et al., Journal of Immunological Methods, Vol. 137(2):167-174 (1991).

Defensins having antimicrobial activity may be capable of reducing the number of living cells of Escherichia coli (DSM 1576) to 1/100 after 8 hours (preferably after 4 hours, more preferably after 2 hours, most preferably after 1 hour, and in particular after 30 minutes) incubation at 20° C. in an aqueous solution of 25% (w/w); preferably in an aqueous solution of 10% (w/w); more preferably in an aqueous solution of 5% (w/w); even more preferably in an aqueous solution of 1% (w/w); most preferably in an aqueous solution of 0.5% (w/w); and in particular in an aqueous solution of 0.1% (w/w) of the defensins having antimicrobial activity.

Defensins having antimicrobial activity may also be capable of inhibiting the outgrowth of Escherichia coli (DSM 1576) for 24 hours at 25° C. in a microbial growth substrate, when added in a concentration of 1000 ppm; preferably when added in a concentration of 500 ppm; more preferably when added in a concentration of 250 ppm; even more preferably when added in a concentration of 100 ppm; most preferably when added in a concentration of 50 ppm; and in particular when added in a concentration of 25 ppm.

Defensins having antimicrobial activity may be capable of reducing the number of living cells of Bacillus subtilis (ATCC 6633) to 1/100 after 8 hours (preferably after 4 hours, more preferably after 2 hours, most preferably after 1 hour, and in particular after 30 minutes) incubation at 20° C. in an aqueous solution of 25% (w/w); preferably in an aqueous solution of 10% (w/w); more preferably in an aqueous solution of 5% (w/w); even more preferably in an aqueous solution of 1% (w/w); most preferably in an aqueous solution of 0.5% (w/w); and in particular in an aqueous solution of 0.1% (w/w) of the defensins having antimicrobial activity.

Defensins having antimicrobial activity may also be capable of inhibiting the outgrowth of Bacillus subtilis (ATCC 6633) for 24 hours at 25° C. in a microbial growth substrate, when added in a concentration of 1000 ppm; preferably when added in a concentration of 500 ppm; more preferably when added in a concentration of 250 ppm; even more preferably when added in a concentration of 100 ppm; most preferably when added in a concentration of 50 ppm; and in particular when added in a concentration of 25 ppm.

The Defensins of the present invention have at least 20%, preferably at least 40%, more preferably at least 50%, more preferably at least 60%, more preferably at least 70%, more preferably at least 80%, even more preferably at least 90%, most preferably at least 95%, and even most preferably at least 100% of the antimicrobial activity of the defensin consisting of the amino acid sequence shown as amino acids 1 to 42 of SEQ ID NO: 2.

Modification: The term “modification” means herein any chemical modification of the defensin. The modification(s) can be substitution(s), deletion(s) and/or insertions(s) of the amino acid(s) as well as replacement(s) of amino acid side chain(s); or use of unnatural amino acids with similar characteristics in the amino acid sequence. In particular the modification(s) can be amidations, such as amidation of the C-terminus.

Identity: The relatedness between two amino acid sequences or between two nucleotide sequences is described by the parameter “identity”.

For purposes of the present invention, the degree of identity between two amino acid sequences is determined by using the program FASTA included in version 2.0x of the FASTA program package (see W. R. Pearson and D. J. Lipman (1988), “Improved Tools for Biological Sequence Analysis”, PNAS 85:2444-2448; and W. R. Pearson (1990) “Rapid and Sensitive Sequence Comparison with FASTP and FASTA”, Methods in Enzymology 183:63-98). The scoring matrix used was BLOSUM50, gap penalty was −12, and gap extension penalty was −2.

The degree of identity between two nucleotide sequences is determined using the same algorithm and software package as described above. The scoring matrix used was the identity matrix, gap penalty was −16, and gap extension penalty was −4.

### DETAILED DESCRIPTION OF THE INVENTION

Defensins

The defensins of the invention may be any antimicrobial peptide recognized by a person skilled in the art as belonging to the defensin class of antimicrobial peptides. The defensins may belong to the alpha-defensin class, the beta-defensin class, the theta-defensin class, the insect defensin class, the fungal defensin class, the mussel defensin class, or other defensin classes wherein the amino acid sequences comprise 6 or 8 cysteines and are structurally similar to any of the before-mentioned defensin classes. The defensins may also be synthetic defensins sharing the characteristic features of any of the defensin classes.

Examples of such defensins include, but are not limited to, those disclosed in PCT applications WO 99/53053, WO 02/085934 or WO 03/044049 (see SEQ ID NO: 2), which are hereby incorporated by reference; or those disclosed in US 60/629,442 (see SEQ ID NO: 2), US 60/632,672 (see SEQ ID NO: 2), US 60/632,673 (see SEQ ID NO: 2), US 60/632,670 (see SEQ ID NO: 2), US 60/632,669 (see SEQ ID NO: 2), US 60/632,486 (see SEQ ID NO: 2) or US 60/642,076 (see SEQ ID NO: 2), which are all incorporated by reference.

In a preferred embodiment a defensin of the invention comprises the amino acid sequence (as represented by the one-letter amino acid code):

(SEQ ID NO: 1)

GFGCNGPWDEDDMQCHNHCKSIKGYKGGYCAKGGFVCKCY

or an amino acid sequence which is at least 60%, preferably 70%, more preferably 80%, even more preferably 90%, and most preferably 95% identical to this sequence. The defensin of the invention may furthermore comprise one or more chemical modifications compared to this amino acid sequence.

The defensins of the invention may also comprise the amino acid sequence:
X1-C-X2-C-X3-C-X4-C-X5-C-X6-C-X7
wherein

X1 represents 0-10 amino acids;

X2 represents 1-15 amino acids;

X3 represents 3-11 amino acids, preferably 3-4 amino acids;

X4 represents 5-12 amino acids;

X5 represents 2-10 amino acids;

X6 represents 0-7 amino acids, preferably 0-1 amino acids; and

X7 represents 0-8 amino acids, preferably 0-5 amino acids.

In an embodiment, the defensin of the invention has antifungal activity. In another embodiment, the defensin of the invention has antibacterial activity. In yet another embodiment, the defensin of the invention has antiviral activity.

A defensin of the invention may be obtained from microorganisms of any genus. For purposes of the present invention, the term “obtained from” as used herein in connection with a given source shall mean that the defensin encoded by a nucleotide sequence is produced by the source or by a strain in which the nucleotide sequence from the source has been inserted. In a preferred aspect, the defensin obtained from a given source is secreted extracellularly.

A defensin of the invention may be a bacterial defensin. For example, the defensin may be a gram positive bacterial defensin such as a Bacillus defensin, e.g., a Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, or Bacillus thuringiensis defensin; or a Streptomyces defensin, e.g., a Streptomyces lividans or Streptomyces murinus defensin; or a gram negative bacterial defensin, e.g., an E. coli or a Pseudomonas sp. defensin.

A defensin of the present invention may also be a fungal defensin, and more preferably a yeast defensin such as a Candida, Kluyveromyces, Pichia, Saccharomyces, Schizosaccharomyces, or Yarrowia defensin; or more preferably a filamentous fungal defensin such as an Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma defensin.

In a preferred aspect, the defensin is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis defensin having antimicrobial activity.

In another preferred aspect, the defensin is an Aspergillus aculeatus, Aspergillus awamori, Aspergillus fumigatus, Aspergillus foetidus, Aspergillus japonicus, Aspergillus nidulans, Aspergillus niger, Aspergillus oryzae, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Trichoderma harzianum, Trichoderma koningii, Trichoderma longibrachiatum, Trichoderma reesei, or Trichoderma viride defensin.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

Furthermore, such defensins may be identified and obtained from other sources including microorganisms isolated from nature (e.g., soil, composts, water, etc.) using the above-mentioned probes. Techniques for isolating microorganisms from natural habitats are well known in the art. The polynucleotide may then be obtained by similarly screening a genomic or cDNA library of another microorganism. Once a polynucleotide sequence encoding a defensin has been detected with the probe(s), the polynucleotide can be isolated or cloned by utilizing techniques which are well known to those of ordinary skill in the art (see, e.g., Sambrook et al., 1989, supra).

Defensins of the present invention also include fused defensins or cleavable fusion defensins in which another defensin is fused at the N-terminus or the C-terminus of the defensin or fragment thereof. A fused defensin is produced by fusing a nucleotide sequence (or a portion thereof encoding another defensin to a nucleotide sequence (or a portion thereof of the present invention. Techniques for producing fusion defensins are known in the art, and include ligating the coding sequences encoding the defensins so that they are in frame and that expression of the fused defensin is under control of the same promoter(s) and terminator.

Infections

Infections which may be treated by the defensins of the invention include all microbial infections, such as viral, fungal and bacterial infections, which are accessible to agents being administered systemically. By infection is meant the pathological state resulting from the invasion of the body by pathogenic microorganisms. The infected body may be a human or animal body. A person skilled in the art of infectious diseases will readily recognize the infections, which are relevant to the present invention.

Examples of such infections include, but are not limited to, Bacteremia and Sepsis (such as Catheter related sepsis, Meninggococcemia, Gonococcemia, Pseudomonas bacteremia, Staphylococcal bacteremia, Bacterial endocarditis); Neonatal and sepsis (such as Early-onset sepsis, Late-onset sepsis); Skin and Soft-tissue infections (such as Impetigo, Cutaneous abscesses, Puncture Wounds, Cellulitis and related skin infections, Necrotizing soft-tissue infections, Infections of deep spaces of neck, Wound infections, Tetanus, Decubitus ulcers); Central Nervous System infections (such as Meningitis, Encephalitis, Brain abscess); Eye infections (such as Eyelid Infections, Infection of the Lacrimal System, Red Eye, Conjunctivitis, Infectious Keratitis (Corneal Ulcers), Uveitis, Orbital and Periorbital infections); Infections of the upper respiratory Tract (such as Common Upper Respiratory syndromes, Otitis Media, Acute Sinusitis, External Otitis); Influenza and infections of the trachea, bronchi and bronchioles (such as Influenza, Laryngitis and Croup (Acute Laryngotracheobronchitis), Epiglottitis, Acute Viral Tracheitis and Tracheobronchitis, Acute Bronchitis and Acute exacerbations of Chronic bronchitis in Adults, Bronchiolitis, Viral Pneumonia); Lower respiratory Tract Infections (such as Pneumonia, Acute community-acquired pneumonia, aspiration pneumonia, atypical pneumonia, legionnaires, legionella micdadel, mellioidosis, Hantavirus pulmonary syndrome, Nosocomial pneumonia, Embolic pneumonia, Staphylococcal pneumonia, Candida in sputum cultures, Pleural effusion versu empyema, Lung abscess, Tuberculosis); Cardiac Infections (such as Infective endocarditis, Endocarditis associated with prosthetic valves, Endocarditis in intravenous Drug users, Pericarditis Myocarditis, Infections of Permanent Cardiac Pacemakers and Implantable cardioverter defibrillators); Gastrointestinal and intraabdominal Infections (such as Gastroenteritis and food Poisoning, Heliobacter pylori and Peptic Ulcer Disease, Gastrointestinal Parasites, Intraabdominal infection, Diverticullitis and related Complications, Billary Tract Infections, Acute Necrotizing Pancreatitis, Pancreatic Abscess, and Infected Pancratic Pseudocyst); Genitourinary Tract Infections (such as Urinary Tract Infections in women, men and elderly, children, Catheter-associated UTI, Fungal UTI, Intrarenal and Perinephric Abscess, Genitorinary Tuberculosis); Sexually transmitted diseases (such as Urethritis, Cervicitis, Genital lesions); Gynecologic and Obstetric Infections (such as Acute Salpingitis, Pelvic Abscesses, Intramniotic Infections, Puerperal Endometritis, Breast Infections); Joint Infections (such as Septic Arthritis, Prosthetic Joint Infections, Infectious Bursitis, Intervertebral Disc Space Infections); Osteomyelitis and diabetic Foot Infections (such as Osteomylitis, Hematogenous osteomyelitis, Contiguous-Focus Osteomyelitis, Diabetic Foot Infections); Infectious Mononucleosis and Mononucleosis-like Syndromes; Infections Due to Fungi, actinomyces, and Nocardia (such as Candidiasis, Histoplasmosis, Cryptococcosis, Aspergillosis, Blastomycosis, Mucormycosis, Sporotrichosis, Actinomycosis, Nocardiosis); Acquired immunodeficiency Syndrome; and Infections in transplantation.

In an embodiment, the infection of the invention is a systemic infection, also referred to as sepsis. By systemic infection is meant the presence of pathogenic microorganisms in tissues or in the blood.

Stability in Human Serum

To assess the stability of the defensins in human serum, according to the invention, a solution of 100 mg/L of a defensin in fresh normal human serum is incubated at 37 degrees Celsius for 0, 1, 3, 6 and 24 hours. Residual activity is measured by performing radial diffusion assays with a sensitive indicator organism (e.g., Staphylococcus carnosus or E. coli); by performing a quantification by HPLC, LCMSMS; or by performing various ELISA techniques well known to a person skilled in the art.

The defensins of the invention may retain at least 90%, preferably 95%, more preferably 97% and most preferably 99% antimicrobial activity after incubation in human serum for one hour at 37 degrees Celsius.

Maximum Tolerable Dose

The maximum tolerable dose is determined according to methods well known to a person skilled in the art of toxicology.

The study, to determine the maximum tolerable dose in mice, should be initiated with a sighting study to select the appropriate dose level for the main study. For example, six dose levels could be investigated using one male and one female mouse at each dose level.

On the basis of any signs of toxicity, the selected dose is administered to five male and five female mice as a single intravenous dose in a dose volume of 10 ml/kg. The dose should be administered slowly in the tail vein (e.g., 20 seconds per injection using a 1 ml syringe with a 25G hypodermic needle). All animals should be observed for any signs of toxicity after 15 minutes, 1 hour, 3 hours, 6 hours and 24 hours after administration. A person skilled in the art of toxicology and animal handling will readily recognize any signs of toxicity revealed by the animals.

The defensins of the invention may have a maximum tolerable dose of at least 50 mg/kg, preferably 75 mg/kg, more preferably 100 mg/kg, and most preferably 125 mg/kg.

Methods and Uses

Formulations of the defensins of the invention are administered to a host suffering from or predisposed to a microbial infection. According to the invention, the defensins are systemic after administration. Generally the dose of the defensins of the invention will be sufficient to decrease the microbial population by at least about 50%, usually by at least 1 log, and may be by 2 or more logs of killing. The defensins of the invention are administered at a dosage that reduces the microbial population while minimizing any side-effects. It is contemplated that the pharmaceutical composition will be obtained and used under the guidance of a physician for in vivo use. The defensins of the invention are particularly useful for killing gram negative bacteria, including Pseudomonas aeruginosa, and Chlamydia trachomatis; and gram-positive bacteria, including various staphylococci and streptococci.

The susceptibility of a particular microbe to killing with the defensins of the invention may be determined by in vitro testing. Typically a culture of the microbe is combined with the antimicrobial polypeptide at varying concentrations for a period of time sufficient to allow the protein to act, usually between about one hour and one day. The viable microbes are then counted, and the level of killing determined.

Microbes of interest include, but are not limited to, Gram-negative bacteria, for example: Citrobacter sp.; Enterobacter sp.; Escherichia sp., e.g., E. coli; Klebsiella sp.; Morganella sp.; Proteus sp.; Providencia sp.; Salmonella sp., e.g., S. typhi, S. typhimurium; Serratia sp.; Shigella sp.; Pseudomonas sp., e.g., P. aeruginosa; Yersinia sp., e.g., Y. pestis, Y. pseudotuberculosis, Y. enterocolitica; Franciscella sp.; Pasturella sp.; Vibrio sp., e.g., V. cholerae, V. parahemolyticus; Campylobacter sp., e.g., C. jejuni; Haemophilus sp., e.g., H. influenzae, H. ducreyi; Bordetella sp., e.g., B. pertussis, B. bronchiseptica, B. parapertussis; Brucella sp., Neisseria sp., e.g., N. gonorrhoeae, N. meningitidis, etc. Other bacteria of interest include Legionella sp., e.g., L. pneumophila; Listeria sp., e.g., L. monocytogenes; Mycoplasma sp., e.g., M. hominis, M. pneumoniae; Mycobacterium sp., e.g., M. tuberculosis, M. leprae; Treponema sp., e.g., T. pallidum; Borrelia sp., e.g., B. burgdorferi; Leptospirae sp.; Rickettsia sp., e.g., R. rickettsii, R. typhi; Chlamydia sp., e.g., C. trachomatis, C. pneumoniae, C. psittaci; Helicobacter sp., e.g., H. pylori, etc.

Non-bacterial pathogens of interest include fungal and protozoan pathogens, e.g., Plasmodia sp., e.g., P. falciparum, Trypanosoma sp., e.g., T. brucei; shistosomes; Entaemoeba sp., Cryptococcus sp., Candida sp., e.g., C. albicans; etc.

Various methods for administration may be employed. The pharmaceutical formulation may be given orally, or may be injected intravascularly, subcutaneously, peritoneally, by aerosol, opthalmically, intra-bladder, topically, etc. For example, methods of administration by inhalation are well-known in the art. The dosage of the therapeutic formulation will vary widely, depending on the specific antimicrobial polypeptide to be administered, the nature of the disease, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered once or several times daily, semi-weekly, etc. to maintain an effective dosage level. In many cases, oral administration will require a higher dose than if administered intravenously. The amide bonds, as well as the amino and carboxy termini, may be modified for greater stability on oral administration. For example, the carboxy terminus may be amidated.

Pharmaceutical Formulations

The defensins of this invention can be incorporated into a variety of pharmaceutical formulations for therapeutic administration. More particularly, the defensins of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, creams, foams, solutions, suppositories, injections, inhalants, gels, microspheres, lotions, and aerosols. As such, administration of the defensins can be achieved in various ways, including oral, buccal, rectal, parenteral, intraperitoneal, intradermal, transdermal, intracheal, etc., administration. According to invention, the defensins are systemic after administration.

The defensins of the invention can be administered alone, in combination with each other, or they can be used in combination with other known compounds (e.g., perforin, anti-inflammatory agents, antibiotics, etc.) In pharmaceutical dosage forms, the defensins may be administered in the form of their pharmaceutically acceptable salts. The following methods and excipients are merely exemplary and are in no way limiting.

For oral preparations, the defensins can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The defensins can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The defensins of the invention can be utilized in aerosol formulation to be administered via inhalation. The defensins can be formulated into pressurized acceptable propellants such as dichlorodifluoromethane, propane, nitrogen and the like.

Furthermore, the defensins can be made into suppositories by mixing with a variety of bases such as emulsifying bases or water-soluble bases. The defensins can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more defensins of the present invention. Similarly, unit dosage forms for injection or intravenous administration may comprise the defensins of the present invention in a composition as a solution in sterile water, normal saline or another pharmaceutically acceptable carrier.

The term “unit dosage form”, as used herein, refers to physically discrete units suitable as unitary dosages for human and animal subjects, each unit containing a predetermined quantity of defensins of the invention calculated in an amount sufficient to produce the desired effect in association with a pharmaceutically acceptable diluent, carrier or vehicle. The specifications for the unit dosage forms of the present invention depend on the particular defensin employed and the effect to be achieved, and the pharmacodynamics associated with the defensin in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Typical dosages for systemic administration range from 0.1 pg to 100 milligrams per kg weight of subject per administration. A typical dosage may be one tablet taken from two to six times daily, or one time-release capsule or tablet taken once a day and containing a proportionally higher content of active ingredient. The time-release effect may be obtained by capsule materials that dissolve at different pH values, by capsules that release slowly by osmotic pressure, or by any other known means of controlled release.

Those of skill will readily appreciate that dose levels can vary as a function of the specific defensin, the severity of the symptoms and the susceptibility of the subject to side effects. Some of the specific defensins are more potent than others. Preferred dosages for a given defensin are readily determinable by those of skill in the art by a variety of means. A preferred means is to measure the physiological potency of a given defensin.

The use of liposomes as a delivery vehicle is one method of interest. The liposomes fuse with the cells of the target site and deliver the contents of the lumen intracellularly. The liposomes are maintained in contact with the cells for sufficient time for fusion, using various means to maintain contact, such as isolation, binding agents, and the like. In one aspect of the invention, liposomes are designed to be aerosolized for pulmonary administration. Liposomes may be prepared with purified proteins or peptides that mediate fusion of membranes, such as Sendai virus or influenza virus, etc. The lipids may be any useful combination of known liposome forming lipids, including cationic or zwitterionic lipids, such as phosphatidylcholine. The remaining lipid will be normally be neutral or acidic lipids, such as cholesterol, phosphatidyl serine, phosphatidyl glycerol, and the like.

For preparing the liposomes, the procedure described by Kato et al. (1991) J. Biol. Chem. 266:3361 may be used. Briefly, the lipids and lumen composition containing peptides are combined in an appropriate aqueous medium, conveniently a saline medium where the total solids will be in the range of about 1-10 weight percent. After intense agitation for short periods of time, from about 5-60 sec., the tube is placed in a warm water bath, from about 25-40° C. and this cycle repeated from about 5-10 times. The composition is then sonicated for a convenient period of time, generally from about 1-10 sec. and may be further agitated by vortexing. The volume is then expanded by adding aqueous medium, generally increasing the volume by about from 1-2 fold, followed by shaking and cooling. This method allows for the incorporation into the lumen of high molecular weight molecules.

Formulations with Other Active Agents

For use in the subject methods, the defensins of the invention may be formulated with other pharmaceutically active agents (such as steroids), which are well-known in the art, particularly other antimicrobial agents. Other agents of interest include a wide variety of antibiotics, as known in the art. Classes of antibiotics include penicillins, e.g., penicillin G, penicillin V, methicillin, oxacillin, carbenicillin, nafcillin, ampicillin, etc.; penicillins in combination with beta-lactamase inhibitors, cephalosporins, e.g., cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; etc.

Anti-mycotic agents are also useful, including polyenes, e.g., amphotericin B, nystatin; 5-flucosyn; and azoles, e.g., miconazol, ketoconazol, itraconazol and fluconazol. Antituberculotic drugs include isoniazid, ethambutol, streptomycin and rifampin. Cytokines may also be included in a formulation of the defensins of the invention, e.g., interferon gamma, tumor necrosis factor alpha, interleukin 12, etc.

The present invention is further described by the following examples which should not be construed as limiting the scope of the invention.

### EXAMPLES

Chemicals used as buffers and substrates were commercial products of at least reagent grade. In the following examples, the Defensin shown as amino acids 1 to 40 of SEQ ID NO: 2 in international patent application WO 03/044049 is referred to as “Plectasin”.

### Example 1

### Stability of Defensin in Human Serum

To assess serum stability, Plectasin (100 mg/L) was incubated at 37° C. in 10% and 90% fresh normal human serum for 0, 1, 3, 6 & 24 hours. Residual activities were measured by performing radial diffusion assays with Staphylococcus carnosus. Full activity was retained even after 24 hours of incubation in 90% human serum.

TABLE 1
Stability in human serum

| | Time (hours) | | | | |
|---|---|---|---|---|---|
| | 0 | 1 | 3 | 6 | 24 |
| Activity (%) 10% serum | 100% | ˜100% | ˜100% | ˜100% | ˜100% |
| Activity (%) 90% serum | 100% | ˜100% | ˜100% | ˜100% | ˜100% |

### Example 2

### Single Dose Intravenous Toxicity Study in the Mouse/Maximum Tolerable Dose

A single intravenous dose was administered slowly in the tail vein (20 seconds per injection) using a 1 ml syringe with a 25G hypodermic needle to 5 male and 5 female mice. All animals were observed for signs of toxicity 15 minutes, 1, 3 and 6 hours after administration and then daily over a period of 14 days. Body weight was recorded on Day 1, 2, 8 and 15. At termination of the study gross necropsy examination of all animals was carried out.

Results (Dose level 125 mg/kg, dose volume 10 ml/kg): 15 minutes after treatment all animals had a discoloration at the injection site. No other signs of toxicity were observed in any of the animals at any observation. The gross necropsy revealed no abnormalities.

### Example 3

### Systemic Availability of Defensin

The in vivo kinetics of plectasin was investigated in NMRI female mice, 25-30 gram (Harlan Scandinavia Aps), after a single 14 mg/kg dose, administered by either intravenous, subcutaneous or intraperitoneal routes. Plectasin concentrations in serum samples were determined 5, 15, 30, 60, 120 and 180 minutes after dosing, using two mice at each time point. At each time point, urine was collected, then mice were anaesthetized with CO₂ and blood collected. The blood samples were centrifuged and serum collected. The concentration of plectasin in serum and urine were determined by LCMSMS.

The maximum observed mean concentrations for i.v., s.c. and i.p. dosing were: 95 mg/L after 5 min., 55 and 34 mg/L after 30 min., respectively. The terminal half-life was estimated to 49-54 min. and the concentration-time data following i.v. bolus administration showed two-compartment behavior (WinNonlin, Pharsight).

TABLE 2
Serum concentration of Plectasin in mg/L

| Time | SC- | SC- | IV- | IV- | IP- | IP- |
|---|---|---|---|---|---|---|
| (min.) | mouse1 | mouse2 | mouse1 | mouse2 | mouse1 | mouse2 |
| 5 | 20.3 | 25.4 | 92.1 | 97.9 | 9.4 | 7.2 |
| 15 | 49.4 | 56.6 | 58.8 | 58.1 | 26.0 | 31.6 |
| 30 | 50.3 | 60.0 | 35.7 | 27.3 | 32.5 | 35.4 |
| 60 | 24.8 | 27.2 | 14.9 | 15.6 | 27.7 | 14.3 |
| 120 | 29.8 | ND | 5.3 | 4.9 | 10.9 | 20.3 |
| 180 | 4.6 | 7.2 | 2.3 | 3.5 | 2.6 | 5.6 |

### Example 4

### Systemic Treatment of Infections with Defensin

(A) Treatment of Pneumococcal Peritoneal Infection.

Three different pneumococcal strains were employed in this study (6A, D39 and 68034). Three mice per sampling point were inoculated with a specific S. pneumoniae i.p., and were treated 1 hour later with a single dose of 10 mg/kg Plectasin. Peritoneal wash was sampled for microbial counts prior to treatment (T=0), and at T=2 and T=5 hrs after. The colony counts are listed below for all three strains. The colony counts in mice treated with Plectasin were significant lower (P<0.0001) compared to the group of vehicle treated mice.

TABLE 3
Strain 68034. Colony counts after 10 mg/kg Plectasin

| | Vehicle | Vehicle | Vehicle | Plectasin | Plectasin | Plectasin |
|---|---|---|---|---|---|---|
| Hours | Mouse1 | Mouse2 | Mouse3 | Mouse1 | Mouse2 | Mouse3 |
| 0 | 3E5 | 5.5E5 | 3.5E5 | 3E5 | 5.5E5 | 3.5E5 |
| 2 | 6E5 | 3.5E5 | 4.5E5 | 3.25E4 | 1.5E4 | 3.25E4 |
| 5 | 7.5E6 | 3.25E6 | 5E6 | 4E2 | 1E2 | 7.5E1 |

TABLE 4
Strain D39. Colony counts after 10 mg/kg Plectasin

| | Vehicle | Vehicle | Vehicle | Plectasin | Plectasin | Plectasin |
|---|---|---|---|---|---|---|
| Hours | Mouse1 | Mouse2 | Mouse3 | Mouse1 | Mouse2 | Mouse3 |
| 0 | 3.25E6 | 1.8E6 | 1.8E6 | 3.25E6 | 1.8E6 | 1.8E6 |
| 2 | 8E6 | 4E6 | 4E6 | 2.75E4 | 1.1E4 | 2E4 |
| 5 | 1.6E7 | 7.25E6 | 6.25E6 | 2.5E2 | 3.5E2 | 1E2 |

TABLE 5
Strain 6A. Colony counts after 10 mg/kg Plectasin

| | Vehicle | Vehicle | Vehicle | Plectasin | Plectasin | Plectasin |
|---|---|---|---|---|---|---|
| Hours | Mouse1 | Mouse2 | Mouse3 | Mouse1 | Mouse2 | Mouse3 |
| 0 | 1.1E6 | 1.55E5 | 1E6 | 1.1E6 | 1.55E6 | 1E6 |
| 2 | 4E7 | 6.75E7 | 2.5E7 | 3.5E5 | 1.78E5 | 3.5E5 |
| 5 | 2.2E8 | 1.45E9 | 8.75E8 | 2.13E4 | 1.43E3 | 1.45E3 |

(B) Survival after Peritoneal Infection with S. pneumoniae D39.

Treatment with Plectasin s.c. 10 mg/kg was initiated one hour after inoculation as described in Example 4A above. The following dosing regimens were investigated in groups of eight mice: vehicle treated, Plectasin once daily (OD) for one day, twice daily (BID) for one day or BID for two days. All mice treated with Plectasin survived the 7-day study period, except for one mouse in the group of BID, one day treatment. The survival fraction of the vehicle treated control group was 0/8 mice (P<0.0002).

(C) Survival after Peritoneal Infection with S. pneumoniae 68034.

Treatment with Plectasin s.c. 10 mg/kg was initiated one hour after inoculation as described in Example 4A above. The following dosing regimens were investigated in groups of eight mice: vehicle treatment, Plectasin BID for one or two days. The survival fraction at seven days post-infection was 6/8 for Plectasin treated mice and 0/8 for vehicle treated mice (P<0.0002).

(D) Treatment of Pneumococcal Pneumonia.

Six to seven mice per sampling point (12 in the no-treatment group) were inoculated with S. pneumoniae (data only shown for strain D39) via the nasopharynx, and 24 hrs later the animals received Plectasin 10 mg/kg or penicillin 30 mg/kg. One day after treatment the animals were necropsied, lungs removed and S. pneumoniae present in lung homogenates were determined and showed in the table below. The data show a significant reduction in the Plectasin and penicillin treatment groups compared to no treatment.

TABLE 6
Strain D39. Colony counts after 10 mg/kg
Plectasin or 30 mg/kg penicillin.

| Start of | | | |
|---|---|---|---|
| treatment | No treatment | Plectasin 10 mg/kg | Penicillin 30 mg/kg |
| 3.06E6 | 3E7 | 3E1 | 4.5E3 |
| 2.18E6 | 2.18E7 | 2.25E3 | 2.25E4 |
| 9.75E5 | 4.8E6 | 1.5E2 | 4.89E5 |
| 1.13E7 | 8.25E5 | 3E1 | 7.5E1 |
| 6.36E6 | 4.8E7 | 5.25E3 | 7.5E1 |
| 1.58E7 | 2.48E7 | 6.75E2 | 7.5E1 |
| | 1.88E7 | 9.75E4 | |
| | 1.13E7 | | |
| | 9E6 | | |
| | 3.75E4 | | |
| | 1.95E6 | | |
| | 3.15E4 | | |

1. A method for treating a microbial infection, comprising contacting a microbial population systemically with a defensin having antimicrobial activity.

2. The method of claim 1, wherein the defensin is capable of retaining at least 90% antimicrobial activity in human serum after one hour at 37 degrees Celsius, and wherein the defensin has a maximum tolerable dose of at least 50 mg/kg in mice.

3. The method of claim 1, wherein the microbial infection is a systemic infection.

4. The method of claim 1, wherein the microbial infection is a bacterial infection and the defensin has antibacterial activity.

5. The method of claim 1, wherein the microbial infection is a fungal infection and the defensin has antifungal activity.

6. A pharmaceutical formulation for systemic administration against a microbial infection, comprising a defensin having antimicrobial activity and a pharmaceutically acceptable carrier.

7. The pharmaceutical formulation of claim 6, wherein the microbial infection is a bacterial infection and the defensin has antibacterial activity.

8. The pharmaceutical formulation of claim 6, wherein the microbial infection is a fungal infection and the defensin has antifungal activity.

9. The pharmaceutical formulation of claim 6, wherein the microbial infection is a systemic infection.

10. The pharmaceutical formulation of claim 6, wherein the defensin is systemic after administration.

11. The pharmaceutical formulation of claim 6, which is a solid composition.

12. The pharmaceutical formulation of claim 11, which is a tablet, capsule or suppository.

13. The pharmaceutical formulation of claim 6, which is a liquid composition.

14. The pharmaceutical formulation of claim 13, which is an injection.

15. The pharmaceutical formulation of claim 6, wherein the pharmaceutically acceptable carrier is suitable for systemic administration.

16. The pharmaceutical formulation of claim 6, which further comprises a second pharmaceutically active agent.

17. The pharmaceutical formulation of claim 16, wherein the second pharmaceutically active agent is an antimicrobial agent.

18. A method for preparing a pharmaceutical formulation for systemic administration against a microbial infection, which method comprises mixing a defensin having antimicrobial activity and a pharmaceutically acceptable carrier.

19. The method of claim 18, wherein the microbial infection is a systemic infection.

20. The method of claim 18, wherein the defensin is systemic after administration.
